(19) United States
(12) Patent Application Publication
(10) Pub. No.: US 2005/0060777 A1
(43) Pub. Date: Mar. 17, 2005

(54) High frequency plant transformation and/or regeneration

(75) Inventors: Luis Lopez-Molina, New York, NY (US); Qi-Wen Niu, New York, NY (US); Nam-Hai Chua, New York, NY (US)

Correspondence Address: ROTHWELL, FIGG, ERNST & MANBECK, P.C., 1425 K STREET, N.W., SUITE 800, WASHINGTON, DC, 20005, US

(73) Assignee: The Rockefeller University, New York, NY (US)

(21) Appl. No.: 10/659,710

(22) Filed: Sep. 11, 2003

(51) Int. Cl. A01H 1/00; C12N 15/82

(52) U.S. Cl. 800/294

(57) ABSTRACT

The present invention is directed to methods for improving Agrobacterium-mediated plant transformation and regeneration of transformed tissue. More specifically, the present invention provides methods for enhancing the frequency of plant transformation by Agrobacterium in which ABI5, ABI5 ortholog protein or ABI5 homolog protein level or activity in transformed plant tissue is suppressed. Any mechanism which suppresses levels or activity of ABI5, ABI5 ortholog protein or ABI homolog protein in plants can be used to enhance the frequency of plant transformation by Agrobacterium. The present invention is further directed to plants, nucleic acids and vectors for use in the methods of invention.

### BACKGROUND OF THE INVENTION

The present invention relates to methods for improving Agrobacterium-mediated plant transformation and/or regeneration of transformed tissue. More specifically, the present invention provides methods for enhancing the frequency of plant transformation by Agrobacterium in which ABI5, ABI5 ortholog protein or ABI5 homolog protein level or activity in transformed plant tissue is suppressed. The present invention also provides methods for enhancing the frequency of regeneration of plants by other methods of plant transformation in which ABI5, ABI5 ortholog protein or ABI5 homolog protein level or activity in transformed plant tissue is suppressed. The present invention further relates to plants, nucleic acids and vectors for use in the methods of invention.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Abscisic acid (ABA) is a phytohormone regulating the initiation and maintenance of seed dormancy. It also plays an essential role in a plant's response to stress, particularly water deprivation, notably by regulating stomatal aperture (Himmelbach et al., 1998). ABA-insensitive screens have been widely used to identify molecular genetic components of the ABA signal transduction pathway (Kornneef et al., 1984; Finkelstein, 1994). In these screens, mutagenized Arabidopsis seeds were exposed to ABA concentrations that inhibit germination of wild type (WT) seeds, and putative mutants that were able to germinate were isolated (Kornneef et al., 1984; Finkelstein, 1994). These screens have allowed the identification of several ABI (ABA insensitive) genes (Lopez-Molina and Chua, 2000; Finkelstein and Lynch, 2000; Gosti et al., 1999; Finkelstein et al., 1998; Leung et al., 1994; Meyer et al., 1994; Giraudat et al., 1992). Recent studies have established that ABI1 and ABI3 are key players in vegetative and embryonic ABA responses, respectively (Himmelbach et al., 1998; Parcy and Giraudat, 1997), and ABI5 is a key player in tolerance acquisition of plants to stress (Lopez-Molina et al., 2001, 2002a).

ABI5 is an Arabidopsis thaliana transcription factor activator belonging to the bZIP family (Finkelstein and Lynch, 2000; Lopez-Molina and Chua, 2000). Arabidopsis contains a small family of ABI5 related factors (Jakoby et al., 2002). ABI5 has homologs in many plant species including dicots such as common bean, soy, tobacco, vine, sunflower and poplar and monocots such as maize, rice and wheat (Kim et al., 1997; Hobo et al., 1999; Davies and Robinson, 2000; Johnson et al., 2002; Sano and Nagata, 2002). All these factors contain three conserved domains (named I, II and III, see FIG. 1) in their N-terminal domain (Finkelstein and Lynch, 2000; Lopez-Molina and Chua, 2000). The basic region, located in the C-terminal part of the protein, is responsible for the DNA binding specificity (Vinson et al., 1989; Shuman et al., 1990). This region has the best conservation (close to 100%) among all these ABI5 bZIP factors (Finkelstein and Lynch, 2000; Lopez-Molina and Chua, 2000). The basic region interacts with ABA responsive elements (ABREs) found in the promoters of stress-responsive genes (Finkelstein et al., 2002). The leucine zipper is just adjacent to the basic region and mediates the homodimerization or heterodimerization of the transcription factor.

ABI5 was initially studied as a key transcriptional regulator in the establishment of an ABA- and osmotic stress-dependent growth arrest taking place during germination, a fragile phase in the life-cycle of a plant (Finkelstein and Lynch, 2000; Lopez-Molina and Chua, 2000; Lopez-Molina et al., 2001). abi5 mutant plants are unable to execute this post-germination growth arrest and are therefore ABA-insensitive (Finkelstein and Lynch, 2000; Lopez-Molina and Chua, 2000). This characteristic phenotype was used to isolate the ABI5 gene by positional cloning (Finkelstein and Lynch, 2000; Lopez-Molina and Chua 2000). Other members (ABFs or AREBs) of the ABI5 gene family in Arabidopsis play a role in ABA and stress responses in vegetative tissues (Choi et al., 2000; Uno et al., 2000; Bensmihen et al., 2002; Kim et al., 2002).

In wild-type Arabidopsis plants, ABI3 expression and activity parallel those described for ABI5 following stratification. During this process, transcript levels of late embryogenesis genes such as AtEm1 and AtEm6 are also re-induced, which might be responsible for the acquired osmotic tolerance in germinated embryos whose growth is arrested. ABI5 expression is greatly reduced in abi3-1 mutants, which has low AtEm1 or AtEm6 expression. Cross complementation experiments showed that 35S-ABI5 could complement abi3-1, whereas 35S-ABI3 cannot complement abi5-4. These results indicate that ABI5 acts downstream of ABI3 to reactivate late embryogenesis program and to arrest growth of germinating embryos. Although ABI5 is consistently located in the nucleus, chromosomal immunoprecipitation (CHIP) experiments revealed that ABA increases ABI5 occupancy on the AtEm6 promoter. (Lopez-Molina et al., 2002b)

It has also been discovered an ABI5-interacting protein, designated as AFP, can form high molecular weight (Mr) complexes with ABI5 in embryo-derived extracts. Like ABI5, ABI five binding protein (AFP) mRNA and protein levels are induced by ABA during seed germination. Two different afp mutant alleles (afp-1 and afp-2) are hypersensitive to ABA, whereas transgenic plants over expressing AFP are resistant. In these plants, AFP and ABI5 protein levels are inversely correlated. Genetic analysis shows that abi5-4 is epistatic to afp-1, indicating the ABA hypersensitivity of afp mutants requires ABI5. Proteasome inhibitor studies show that ABI5 stability is regulated by ABA through ubiquitin-related events. When expressed together, AFP and ABI5 are colocalized in nuclear bodies, which also contain COP1, a RING motif protein. These results suggest that AFP attenuates ABA signals by targeting ABI5 for ubiquitin-mediated degradation in nuclear bodies. (Lopez-Molina et al., 2003)

Many plant transformation and regeneration systems have been developed. These systems have varying degrees of efficiency, including varying levels of transformation and regeneration frequencies. It is appreciated in the field that there remains a need for developing methods to improve Agrobacterium-mediated transformation of plants, including regeneration of transformed tissue.

### SUMMARY OF THE INVENTION

The present invention relates to methods for improving Agrobacterium-mediated plant transformation and/or regeneration of transformed tissue. More specifically, the present invention provides methods for enhancing the frequency of plant transformation by Agrobacterium in which ABI5, ABI5 ortholog protein or ABI5 homolog protein level or activity in transformed plant tissue is suppressed. The present invention also provides methods for enhancing the frequency of regeneration of plants by other methods of plant transformation in which ABI5, ABI5 ortholog protein or ABI5 homolog protein level or activity in transformed plant tissue is suppressed. The present invention further relates to plants, nucleic acids and vectors for use in the methods of invention.

As described herein, it has been discovered that ABI5 plays a key role in decreasing frequency of Agrobacterium tumefaciens-mediated transformation of Arabidopsis thaliana. Specifically, it has been discovered that Arabidopsis transformation efficiency is directly dependent on ABI5 protein. Reduced level or activity of ABI5 protein results in higher efficiency of Agrobacterium-mediated transformation of Arabidopsis. ABI5 has homologs in many plant species, and the reduction of protein levels or activity of these ABI5 homologs also leads to enhanced efficiency of Agrobacterium-mediated transformation of these plant species. Any mechanism which suppresses protein level or activity of ABI5, ABI5 ortholog protein or ABI homolog protein in a plant species can be used to enhance the frequency of plant transformation by Agrobacterium.

In one aspect of the invention, transgenic plants with low endogenous ABI5 and/or ABI5 homolog or ortholog protein levels are provided as target plants for stable transformation by Agrobacterium tumefaciens. According to this aspect, a plant with low endogenous ABI5, ABI5 homolog or ABI5 ortholog protein levels in a target crop species is established. Any method culminating in the down regulation of ABI5, ABI5 orthologs or ABI5 homologs expression or activity (mRNA and protein) can be used to provide a plant with low endogenous protein levels leading to an increase in plant transformation efficiency. The methods include expression of transgenic or non-transgenic nucleic acids and proteins leading to a down regulation of ABI5, ABI5 orthologs or ABI5 homologs expression and activity (mRNA and protein). Some specific examples include, but are not limited to, T-DNA or transposon insertions in ABI5, ABI5 orthologs or ABI5 homologs, RNA interference techniques that down regulate ABI5, ABI5 orthologs or ABI5 homologs, transgenic plants over expressing negative regulators of ABI5, ABI5 orthologs or ABI5 homologs protein levels, sense or co-suppression, antisense and ribozymes. Suitable vectors and techniques for preparing transgenic plants are well known to a skilled artisan. These techniques include transformation with DNA employing A. tumefaciens or A. rhizogenes as the transforming agent, electroporation, particle acceleration, etc. See for example, U.S. Pat. No. 6,608,242, incorporated herein by reference.

Alternatively, a plant with low endogenous protein levels or activity of a protein that modulates the expression of ABI5, ABI5 orthologs or ABI5 homologs is established. One example of such a modulating protein is ABI3, ABI3 ortholog or ABI3 homolog. Any method culminating in the down regulation of ABI3, ABI3 orthologs or ABI3 homologs expression or activity (mRNA and protein) can be used to provide a plant with low endogenous protein levels leading to an increase in plant transformation efficiency. The methods include expression of transgenic or non-transgenic nucleic acids and proteins leading to a down regulation of ABI3, ABI3 orthologs or ABI3 homologs expression and activity (mRNA and protein). Some specific examples include, but are not limited to, T-DNA or transposon insertions in ABI3, ABI3 orthologs or ABI3 homologs, RNA interference techniques that down regulate ABI3, ABI3 orthologs or ABI3 homologs, transgenic plants over expressing negative regulators of ABI3, ABI3 orthologs or ABI3 homologs protein levels, sense or co-suppression, antisense and ribozymes. Suitable vectors and techniques for preparing transgenic plants are well known to a skilled artisan. These techniques include transformation with DNA employing A. tumefaciensor A. rhizogenes as the transforming agent, electroporation, particle acceleration, etc. See for example, U.S. Pat. No. 6,608,242, incorporated herein by reference.

The transgenic plants according to this first embodiment of the invention are then used for Agrobacterium-mediated transformation using standard techniques in the art to insert DNA sequences of interest into the genome of the plant species. In general an Agrobacterium vector containing the desired DNA sequences is used to insert DNA sequences of interest into the genome of the plant species.

In a second aspect of the invention, plant transformation vectors containing DNA sequences designed to down regulate ABI5, ABI5 homolog or ABI5 ortholog protein levels in the host target cell are provided. Any vector useful for Agrobacterium-mediated plant transformation is useful in this aspect of the invention. Such vectors are well known to a skilled artisan. See for example, U.S. Pat. No. 6,608,242, incorporated herein by reference. According to this aspect, a target crop, which may be a transgenic or non-transgenic crop species, is used directly as a target for stable transformation by Agrobacterium tumefaciens. A plant transformation vector can be designed so as to include genes or other sequences that can down regulate ABI5, ABI5 ortholog or ABI5 homolog activity and/or protein levels in infected cells. This down regulation can be achieved, for example, by using DNA sequences encoding ABI5 (or DNA sequences encoding a homolog or ortholog) mutants without the activation domain. This type of mutants functions as dominant negatives because they form inactive heterodimers with endogenous wild type ABI5 thus interring with the activity of the latter. Alternatively, hairpin RNA sequences homologous to ABI5 coding sequences, ABI5 homolog coding sequences or ABI5 ortholog coding sequences can be used to reduce expression of ABI5, ABI5 homolog and ABI5 ortholog genes in target cells.

Alternatively, plant transformation vectors containing DNA sequences designed to down regulate a protein that modulates the expression of ABI5, ABI5 homolog or ABI5 ortholog protein levels in the host target cell are provided. Any vector useful for Agrobacterium-mediated plant transformation is useful in this aspect of the invention. Such vectors are well known to a skilled artisan. See for example, U.S. Pat. No. 6,608,242, incorporated herein by reference. One example of such a modulating protein is ABI3, ABI3 ortholog or ABI3 homolog. According to this aspect, a target crop, which may be a transgenic or non-transgenic crop species, is used directly as a target for stable transformation by Agrobacterium tumefaciens. A plant transformation vector can be designed so as to include, but not limited to, genes or other sequences that can down regulate ABI3, ABI3 ortholog or ABI3 homolog activity and/or protein levels in infected cells, including down regulating ABI3 mRNA levels. This down regulation can be achieved by, for example, using DNA sequences encoding ABI3 (or DNA sequences encoding a homolog or ortholog) mutants lacking appropriate ABI3 subdomains. This type of mutants functions as dominant negatives because they form inactive heterodimers with endogenous wild type ABI3 (or homolog or ortholog) or non-productive complexes with ABI3-interacting proteins, thus interfering with the activity of ABI3. Alternatively, hairpin RNA sequences homologous to ABI3 coding sequences, ABI3 homolog coding sequences or ABI3 ortholog coding sequences can be used to reduce expression of ABI3, ABI3 homolog and ABI3 ortholog genes in target cells.

The Agrobacterium vectors of this second embodiment can also contain DNA sequences of interest in order to insert such sequences into the genome of the plant species by Agrobacterium-mediated transformation using standard techniques in the art. Alternatively, a second Agrobacterium vector containing the desired DNA sequences can be used to insert DNA sequences of interest into the genome of the plant species.

### BRIEF DESCRIPTION OF THE FIGURES

FIG. 1 is a schematic of the ABI5 transcription factor activator homologs showing the three conserved domains in their N-terminal domain and the basic DNA binding and leucine zipper domains in the C-terminal domain.

FIG. 2 is a schematic of the binary vector pER16.

FIG. 3 shows ABI5 expression in floral tissue during and after vacuum infiltration with Agrobacterium.

FIG. 4 shows the root transformation efficiency after 40 days culture in selection medium for wild type (WS) plant material, abi5-4 mutant plant material and 35S-ABI5 plant material.

FIG. 5 shows shoot regeneration index of Arabidopsis root explants wild type (Ler or WS) plant material, abi3-1 mutant plant material and 35S-ABI3 plant material.

FIG. 6 schematically shows an example of a vector according to a preferred embodiment of the present invention. The vector contains a nucleic acid sequence that is useful for reducing ABI5 protein levels in transformed cells to enhance transformation and/or regeneration. The vector also contains elements for subsequently deleting the nucleic acid sequence.

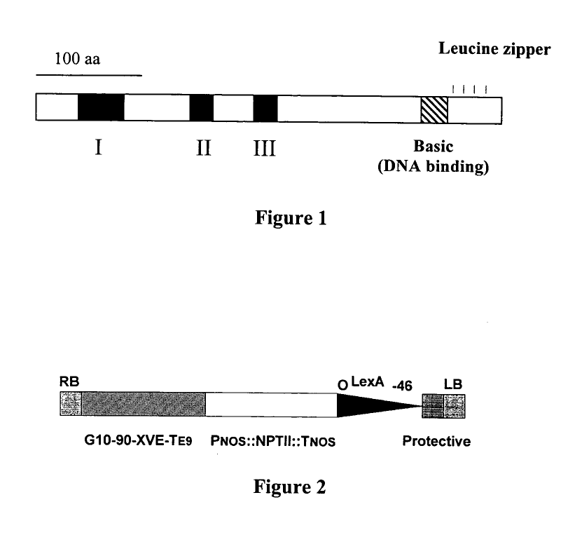

### DETAILED DESCRIPTION OF THE INVENTION

The present invention is directed to methods for improving Agrobacterium-mediated plant transformation and/or regeneration of transformed tissue. More specifically, the present invention provides methods for enhancing the frequency of plant transformation by Agrobacterium in which ABI5, ABI5 ortholog protein or ABI5 homolog protein level or activity in transformed plant tissue is suppressed. As described herein in further detail, any mechanism that suppresses levels or activity of ABI5, ABI5 ortholog protein or ABI homolog protein in plants can be used to enhance the frequency of plant transformation by Agrobacterium. The present invention also provides methods for enhancing the frequency of regeneration of plants by other methods of plant transformation in which AB5, ABI5 ortholog protein or ABI5 homolog protein level or activity in transformed plant tissue is suppressed. As described herein in further detail, any mechanism that suppresses levels or activity of ABI5, ABI5 ortholog protein or ABI homolog protein in plants can be used to enhance the frequency of plant regeneration of transformed plant tissue. The present invention is further directed to plants, nucleic acids and vectors for use in the methods of invention.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982); Sambrook et al., Molecular Cloning: A Laboratory Manual, 2^{nd} Edition, Cold Spring Harbor Laboratory Press, New York (1989); Sambrook and Russell, Molecular Cloning: A Laboratory Manual, 3^{rd} Edition, Cold Spring Harbor Laboratory Press, New York (2001); Ausubel et al., Current Protocols in Molecular Biology, John Wiley and Sons, New York (1992); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994); Arabidopsis protocols, J. M. Martinez-Zapater and J. Salinas, Eds., Human Press, Totowa, N.J., USA (2001) and the various references cited therein.

The following definitions are used for the present invention.

The term “plant” includes whole plants, plant organs (e.g., leaves, stems, roots, etc.), seeds and plant cells (in planta or in tissue culture) and progeny of same. The class of plants which can be used in the methods of the invention is generally as broad as the class of higher plants amenable to Agrobacterium transformation techniques, including both monocotyledonous and dicotyledonous plants.

The term “seed” or “seedling” includes all stages in the life of a plant and includes somatic embryos and primed seeds.

A seed, seedling or plant which has a reduced protein level or activity of ABI5, ABI5 homolog or ABI5 ortholog is one which produces less AB5, ABI5 homolog or ABI ortholog protein level or activity as compared to a wild type seed, seedling or plant, respectively, under identical conditions. It has been found that any reduction in ABI5, ABI5 homolog or ABI ortholog protein level or activity as compared to a wild type results in an enhance frequency of transformation and/or regeneration.

As used herein, the term “enhancement” means increasing the frequency of transformation in methods of Agrobacterium-mediated transformation or the frequency of regeneration of transformed plants in other methods of plant transformation. In order to achieve enhancement of the frequency of transformation and/or regeneration, the protein level or activity of ABI15, ABI5 homolog or ABI5 ortholog is reduced in plant cells or tissue as described in further detail herein. The frequency of transformation and/or regeneration is measured by the number of transformed plants produced. An enhanced frequency of transformation and/or regeneration is one which is about 1.5-fold greater or more compared to other Agrobacterium-mediated plant transformation in which the protein level or activity of ABI5, ABI5 homolog or ABI5 ortholog is not reduced in plant cells or tissue. For example, as described herein, enhanced frequencies of Agrobacterium-mediated transformation of 1.8 fold was achieved for root transformation and 11.3 fold was achieved for in planta infiltration.

Agrobacterium-mediated transformation means techniques for producing transformed plants using Agrobacterium tumefaciens. The class of plants amenable to Agrobacterium-mediated transformation techniques is generally as broad as the class of higher plants, including both monocotyledonous and dicotyledonous plants. Techniques for transforming plants with Agrobacterium, including the preparation of vectors for use in such transformation, are well known to a skilled artisan.

The term “isolated” means separated from its natural environment.

The term “polynucleotide” refers in general to a nucleotide sequence such as a nucleic acid fragment. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, or synthetic DNA. An isolated polynucleotide useful in the present invention may include less than the full coding sequence. For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, translation leader sequences, introns, and polyadenylation recognition sequences. Promoters may be constitutive, inducible or derepressible. Examples of inducible promoters include but are not limited to the promoters described by Zuo et al. (2002b), Chua and Aoyama (2000), Chua and Aoyama (2001), Zuo et al. (2003) and Horvath et al. (2002).

The term “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into polypeptide by the cell. “cDNA” refers to a double-stranded DNA that is complementary to and derived from mRNA. “Sense” RNA refers to an RNA transcript that includes portions of the mRNA. “Antisense RNA” refers to an RNA transcript that is complementary to all or part of a target primary transcript or mRNA and that blocks the expression of a target gene (see, e.g., U.S. Pat. No. 5,107,065). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, hairpin RNA (RNAi; Chuang and Meyerowitz, 2000; Waterhouse et al., 1998; Guo et al., 2003) or other RNA that may not be translated but yet has an effect on cellular processes.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to nucleic acid fragment, means that the component nucleotides were assembled in vitro. Manual chemical synthesis of nucleic acid fragments may be accomplished using well established procedures, or automated chemical synthesis can be performed using one of a number commercially available machines. Accordingly, the nucleic acid fragments can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “polypeptides” is to be understood to mean peptides or proteins that contain two or more amino acids which are bound via peptide bonds.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

A transgenic plant having low endogenous ABI5, ABI5 ortholog or ABI5 homolog protein levels or activity refers to a plant having a modified genome such that the protein level or activity of ABI5, ABI5 ortholog or ABI5 homolog is reduced compared to a wild type plant. The genome can be stably modified to produce transgenic plants using techniques well known to a skilled artisan and as exemplified herein.

Homology, sequence similarity or sequence identity of nucleotide or amino acid sequences may be determined conventionally by using known software or computer programs such as the BestFit or Gap pair wise comparison programs (GCG Wisconsin Package, Genetics Computer Group, 575 Science Drive, Madison, Wis. 53711). Best Fit uses the local homology algorithm of Smith and Waterman (1981), to find the best segment of identity or similarity between two sequences. Gap performs global alignments: all of one sequence with all of another similar sequence using the method of Needleman and Wunsch (1970). When using a sequence alignment program such as Best Fit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores. These programs are often used to identify homologs and orthologs of a given protein.

As shown herein, it was found that ABI5 plays a key role in decreasing the frequency of Agrobacterium tumefaciens-mediated transformation of Arabidopsis thaliana. Indeed, it was observed that Arabidopsis transformation efficiency is directly dependent on ABI5 protein levels. Transformation efficiency of abi5 mutant plants by vacuum infiltration with Agrobacteria is, on average, 10 times of wild type Arabidopsis. While transformation efficiency of Arabidopsis transgenic plants carrying a 35S-ABI5 is only about 30% of wild type. It was found that ABI5 is expressed in ovules, which are the target tissues for Arabidopsis plant transformation by vacuum infiltration with Agrobacteria (Ye et al., 1999), and the treatment of plants by vacuum infiltration clearly upregulate(s) the expression of ABI5 in ovules. These results indicate that there is an inverse correlation between ABI5 expression levels in ovules and transformation efficiency. Reduction of ABI5 levels or activity increases transformation frequency.

Similar results were also found in root transformation assays. A prerequisite for high transformation efficiency in root assays is to have efficient shoot regeneration. The results show that shoot regeneration is also inversely correlated with ABI5 protein levels. The present discovery that transformation efficiency is inversely related to ABI5 levels is also consistent with the notion that ABI5 and ABI5-like transcription factors are stress factors operating to protect the plant from abiotic and biotic stresses (Choi et al., 2000; Uno et al., 2000; Bensmihen et al., 2002; Kim et al., 2002). When ABI5 is absent or downregulated, the plant becomes more sensitive to stress resulting in a higher transformation efficiency upon Agrobacterium infection.

These results demonstrate that down regulating ABI5 levels or its activity facilitates stable transformation mediated by Agrobacterium tumefaciens in Arabidopsis. ABI5 orthologs or homologs are found in many plant species including species of agricultural importance such as wheat, rice, beans, sunflower, maize, etc. Therefore, the ABI5 orthologs and/or ABI5 homologs will have a similar function in regulating regeneration and transformation efficiency in these species. Examples of such homologs and/or orthologs are disclosed in Kim et al. (1997), Hobo et al. (1999), Davies and Robinson (2000), Johnson et al. (2002) and Sano and Nagata (2002). ABI5 homologs and orthologs can also be identified using the above-identified computer programs or others known in the art. Examples of ABI5 homologs or orthologs identified in this manner include, but are not limited to, the following Genbank accession numbers with respect to the protein accession: AAK39132 (Phaseolus vulgaris), AAM75354 (Triticum aestivum), AAM75355 (Triticum aestivum), BAC10342 (Oryza sativa), BAB89789 (Oryza sativa), AAF65459 (Oryza sativa), T04353 (Oryza sativa), AAL86016 (Oryza sativa) and AAG01025 (Populus balsamifera subsp. trichocarpa×Populus deltoids).

In addition, it was found that Agrobacterium transformation efficiency can be enhanced by modulating the activity of proteins that modulate the protein level or activity of ABI5, ABI5 orthologs or ABI5 homologs. One such protein is ABI3. ABI3 orthologs or homologs are found in many plant species including species of agricultural importance such as wheat, rice, beans, sunflower, maize, etc. Therefore, the ABI3 orthologs and/or ABI3 homologs will have a similar function in regulating regeneration and transformation efficiency in these species. Examples of such homologs and/or orthologs are disclosed in Hobo et al. (1999) and Lopez-Molina and Chua (2000) for rice, Cahoon et al. (2003) for momordica, maize and rice, Bobb et al. (1995) for common bean, Rhode et al. (2002) for poplar, Shiota et al. (1998) for carrot, Lazarova et al. (2002) for cedar and Noel et al. (2003) for potato (Solanum tuberosum). ABI3 homologs and orthologs can also be identified using the above-identified computer programs or others known in the art. Examples of ABI3 homologs or orthologs identified in this manner include, but are not limited to, the following Genbank accession numbers with respect to the protein accession: AAA87030 (Phaseolus vulgaris), CAC19186 (Chamaecyparis nootkatensis), BAA82596 (Daucus carota) and CAC84597/CAC84596 (Solanum tuberosum).

Several methods can be used to diminish ABI5 and ABI5 homolog or ortholog protein level or activity in plants, which methods are well known to a skilled artisan.

In one aspect of the invention, transgenic plants with low endogenous ABI5 and/or ABI5 homolog or ortholog protein levels are provided as target plants for stable transformation by Agrobacterium tumefaciens. According to this aspect, a plant with low endogenous ABI5, ABI5 homolog or ABI5 ortholog protein levels in a target crop species is established. Any method culminating in the down regulation of ABI5, ABI5 orthologs or ABI5 homologs expression or activity (mRNA and protein) can be used to provide a plant with low endogenous protein levels leading to an increase in plant transformation efficiency. The methods include expression of transgenic or non-transgenic nucleic acids and proteins leading to a down regulation of ABI5, ABI5 orthologs or ABI5 homologs expression and activity (mRNA and protein). Some specific examples include, but are not limited to, T-DNA or transposon insertions in ABI5, ABI5 orthologs or ABI5 homologs, RNA interference techniques that down regulate ABI5, ABI5 orthologs or ABI5 homologs, transgenic plants over expressing negative regulators of ABI5, ABI5 orthologs or ABI5 homologs protein levels, sense or co-suppression, antisense and ribozymes. Suitable vectors and techniques for preparing transgenic plants are well known to a skilled artisan. These techniques include transformation with DNA employing A. tumefaciens or A. rhizogenes as the transforming agent, electroporation, particle acceleration, etc. See for example, U.S. Pat. No. 6,608,242, incorporated herein by reference.

Alternatively, a plant with low endogenous protein levels or activity of a protein which modulates the expression of ABI5, ABI5 orthologs or ABI5 homologs is established. As shown herein, ABI5, ABI5 homolog or ABI5 ortholog protein levels can be reduced by modulating protein level or activity of proteins which modulate the protein level or activity of ABI5, ABI5 homolog or ABI5 ortholog. A protein that modulates the protein level of ABI5, ABI5 ortholog or ABI5 homolog includes, but is not limited to ABI3, ABI3 homolog or AB3 ortholog. ABI3 homologs or orthologs are known or can be identified as described above. Any method culminating in the down regulation of ABI3, ABI3 orthologs or ABI3 homologs expression or activity (mRNA and protein) can be used to provide a plant with low endogenous protein levels leading to an increase in plant transformation efficiency. The methods include expression of transgenic or non-transgenic nucleic acids and proteins leading to a down regulation of ABI3, ABI3 orthologs or ABI3 homologs expression and activity (mRNA and protein). Some specific examples include, but are not limited to, T-DNA or transposon insertions in ABI3, ABI3 orthologs or ABI3 homologs, RNA interference techniques that down regulate ABI3, ABI3 orthologs or ABI3 homologs, transgenic plants over expressing negative regulators of ABI3, ABI3 orthologs or ABI3 homologs protein levels, sense or co-suppression, antisense and ribozymes. Suitable vectors and techniques for preparing transgenic plants are well known to a skilled artisan. These techniques include transformation with DNA employing A. tumefaciens or A. rhizogenes as the transforming agent, electroporation, particle acceleration, etc. See for example, U.S. Pat. No. 6,608,242, incorporated herein by reference.

The transgenic plants according to this first embodiment of the invention are then used for Agrobacterium-mediated transformation using standard techniques in the art to insert DNA sequences of interest into the genome of the plant species. In general an Agrobacterium vector containing the desired DNA sequences is used to insert DNA sequences of interest into the genome of the plant species.

In a second embodiment, plant transformation vectors containing DNA sequences designed to down regulate ABI5, ABI5 homolog or ABI5 ortholog protein levels in the host target cell are provided. Any vector useful for Agrobacterium-mediated plant transformation are useful in this aspect of the invention. Such vectors are well known to a skilled artisan. See for example, U.S. Pat. No. 6,608,242, incorporated herein by reference. According to this aspect, a target crop, which may be a transgenic or non-transgenic crop species, is used directly as a target for stable transformation by Agrobacterium tumefaciens. A plant transformation vector can be designed so as to include genes or other sequences that can down regulate ABI5, ABI5 ortholog or ABI5 homolog activity and/or protein levels in infected cells. Plant transformation vectors can be prepared using techniques well known to a skilled artisan. The down regulation of ABI5, ABI5 ortholog or ABI5 homolog activity and/or activity can be achieved using techniques well known to a skilled artisan. For example, and non-limiting, such down regulation can be achieved by using DNA sequences encoding ABI5 (or DNA sequences encoding a homolog or ortholog) mutants without the activation domain. This type of mutants functions as dominant negatives because they form inactive heterodimers with endogenous wild type ABI5 (or homolog or ortholog) thus interfering with the activity of the latter. Alternatively, hairpin RNA sequences (Waterhouse et al., 1998; Chuang and Meyerowitz, 2000; Guo et al., 2003) homologous to ABI5 coding sequences, ABI5 homolog coding sequences or ABI5 ortholog coding sequences can be used to reduce expression of ABI5, ABI5 homolog and ABI5 ortholog genes in target cells.

Alternatively, plant transformation vectors containing DNA sequences designed to down regulate a protein that modulates the expression of ABI5, ABI5 homolog or ABI5 ortholog protein levels in the host target cell are provided. As shown herein, ABI5, ABI5 homolog or ABI5 ortholog protein levels can be reduced by modulating protein level or activity of proteins that modulate the protein level or activity of ABI5, ABI5 homolog or ABI5 ortholog. A protein that modulates the protein level of ABI5, ABI5 ortholog or ABI5 homolog includes, but is not limited to, ABI3, ABI3 homolog or ABI3 ortholog. Any vector useful for Agrobacterium-mediated plant transformation are useful in this aspect of the invention. Such vectors are well known to a skilled artisan. See for example, U.S. Pat. No. 6,608,242, incorporated herein by reference. According to this aspect, a target crop, which may be a transgenic or non-transgenic crop species, is used directly as a target for stable transformation by Agrobacterium tumefaciens. A plant transformation vector can be designed so as to include, but not limited to, genes or other sequences that can down regulate ABI3, ABI3 ortholog or AB13 homolog activity and/or protein levels in infected cells. Binary vectors can be prepared using techniques well known to a skilled artisan. The down regulation of AB13, AB13 ortholog or AB13 homolog level and/or activity, including ABI3 mRNA levels, can be achieved using techniques well known to a skilled artisan. This down regulation can be achieved by, for example, using DNA sequences encoding ABI3 (or DNA sequences encoding a homolog or ortholog) mutants lacking appropriate AB13 subdomains. This type of mutants functions as dominant negatives because they form inactive complexes with AB13-interacting proteins thus interfering with the activity of ABI3. Alternatively, hairpin RNA sequences (Waterhouse et al., 1998; Chuang and Meyerowitz, 2000; Guo et al., 2003) homologous to ABI3 coding sequences, ABI3 homolog coding sequences or ABI3 ortholog coding sequences can be used to reduce expression of ABI3, ABI3 homolog and ABI3 ortholog genes in target cells.

The Agrobacterium vectors of this second embodiment can also contain DNA sequences of interest in order to insert such sequences into the genome of the plant species by Agrobacterium-mediated transformation using standard techniques in the art. Alternatively, a second Agrobacterium vector containing the desired DNA sequences can be used to insert DNA sequences of interest into the genome of the plant species.

In a preferred embodiment, a gene cassette is used which allows for the activation and removal of a transgene or nucleic acid sequence in transgenic plants or regenerated shoots. For example, the gene cassette allows the reduction of ABI5 (or homolog or ortholog) protein level in transformed cells in accordance with the above aspects of the invention, which includes the various mechanisms for reducing ABI5 (or homolog or ortholog) protein level or activity, including by reducing ABI3 (or homolog or ortholog) protein level or activity. As described herein, the reduced ABI5 (or homolog or ortholog) protein level results in an increased frequency of transformation and/or regeneration. The gene cassette further allows the deletion of the nucleic acid sequence responsible for the reduction in ABI5 (or homolog or ortholog) protein levels in transformed cells. Examples of gene cassettes which can be used in this embodiment are described in Moller et al. (2001) and Zuo et al. (2001). This preferred embodiment is particularly advantageous because reduction of ABI5 (or homolog or ortholog) or of ABI3 (or homolog or ortholog) protein level in transgenic plants may have an unwanted impact on agronomic traits. This preferred embodiment obviates this potential problem. In addition, this preferred embodiment also eliminates other unwanted DNA sequences used in the construct, thus leaving only the trait gene intact. This preferred embodiment is further described with reference to the following non-limiting example.

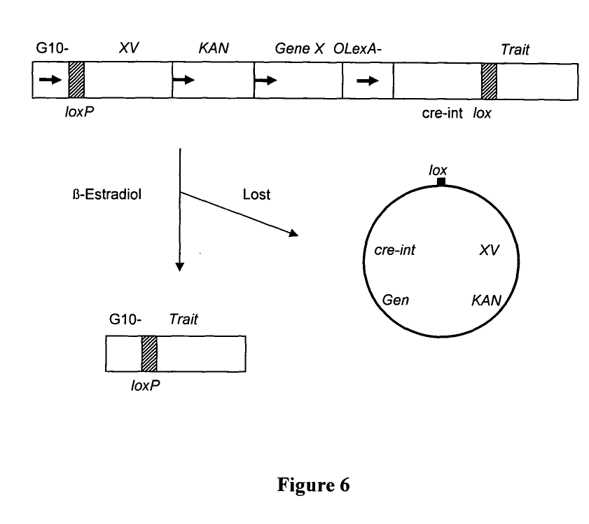

Reduced ABI5 protein levels in transformed cells is accomplished using modifications of the CLX vector (Zuo et al., 2001). These modifications are shown schematically in FIG. 6. Four transcription units are located within the two loxP sites, and downstream of the second loxP site, the trait gene is terminated by the rbcS3A polyadenylation sequence. According to this example, the four transcription units are:

XVE consists of the coding sequence of the XVE hybrid transactivator terminated by the rbcsE9 polyA addition sequence. XVE is activated by the G10-90 promoter upstream of the loxP site.

(2) KAN consists of the nopaline synthase (NOS) gene promoter, the coding sequence of the neomycin phosphotransferase (NPT) II, and the NOS polyadenylation sequence.

(3) cre-int consists of 8 copies of the LexA operator sequence fused to the -46 CaMV 35S prmoter, the coding sequence of Cre interrupted by an intron and terminated by the NOS polyadenylation sequence. The LexA operator sequence is used as a DNA binding site for the XVE transcription activator.

(4) Gene X consists of a 35S promoter or any other appropriate promoter to drive the expression of a DNA sequence flanked by an rbcs3C polyadenylation sequence. The DNA sequence may encode a dominant negative form of ABI5 or any other sequences (e.g., RNAi) as described herein that can reduce ABI5 expression levels.

This modified CLX vector is used to transform plant cells and transgenic plants are identified by their growth on a kanamycin selection medium. Expression of Gene X results in an increased transformation and/or regeneration frequency because of the resulting reduced ABI5 protein level or activity in transformed cells. Transgenic plants or regenerated shoots are then treated with an inducer, e.g. β-estradiol, to induce the XVE hybrid transcription factor allowing the latter to translocate into the nucleus to activate the Cre transcription unit. Expression of Cre in the transgenic plants or regenerated shoots leads to DNA recombination at the loxP sites and excision of the 4 transcription units flanked by the two loxP sites, resulting in the constitution of the G10-90/trait gene transcription unit which is now active.

In accordance with the present invention, transgenic plants can be recovered at a higher frequency than has been achieved prior to the present invention using Agrobacterium-mediated transformation. These transgenic plants can be prepared to express various phenotypes of agronomic interest. Such genes include, but are not limited to, those described herein. DNA sequences and vectors directed to such genes are prepared using techniques well known to a skilled artisan. Such genes can be prepared with appropriate regulatory regions as may be desired for expression of the gene in the transgenic plant as is well known to a skilled artisan.

1. Genes that Confer Resistance or Tolerance to Pests or Disease

Plant disease resistance genes. Plant defenses are often activated by specific interaction between the product of a disease resistance (R) gene in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium fulvum (Jones et al. (1994)), the tomato Pto gene, which encodes a protein kinase, for resistance to Pseudomonas syringae pv. tomato (Martin et al. (1993)), and the Arabidopsis RSSP2 gene for resistance to Pseudomonas syringae (Mindrinos et al. (1994)).

(B). A Bacillus thuringiensis protein, a derivative thereof or a synthetic polypeptide modeled thereon, such as, a nucleotide sequence of a Bt δ-endotoxin gene (Geiser et al. (1986)). Moreover, DNA molecules encoding δ-endotoxin genes can be purchased from American Type Culture Collection (Rockville, Md.), under ATCC accession numbers. 40098, 67136, 31995 and 31998.

(C) A lectin, such as nucleotide sequences of several Clivia miniata mannose-binding lectin genes (Van Damme et al. (1994)).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al. (1987)), a tobacco proteinase inhibitor I (Huub et al. (1993)), and an α-amylase inhibitor (Sumitani et al. (1993)).

(F) An insect-specific peptide or neuropeptide which, upon expression, disrupts the physiology of the affected pest. Examples of such genes include, an insect diuretic hormone receptor (Regan (1994), an allostatin identified in Diploptera puntata (Pratt (1989)), insect-specific, paralytic neurotoxins (U.S. Pat. No. 5,266,361).

(G) An insect-specific venom produced in nature by a snake, a wasp, etc., such as, a scorpion insectotoxic peptide (Pang (1992)).

(H) An enzyme responsible for a hyperaccumulation of a monoterpene, a sesquiterpene, a steroid, hydroxamic acid, a phenylpropanoid derivative or another non-protein molecule with insecticidal activity.

(I) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al. (1993)) and parsley ubi4-2 polyubiquitin gene (Kawalleck et al. (1993)).

(J) A molecule that stimulates signal transduction. Examples of such molecules include, nucleotide sequences for mung bean calmodulin cDNA clones (Botella et al. (1994)), a nucleotide sequence of a maize calmodulin cDNA clone (Griess et al. (1994)).

(K) A hydrophobic moment peptide. See U.S. Pat. Nos. 5,659,026 and 5,607,914, the latter teaches synthetic antimicrobial peptides that confer disease resistance.

(L) A membrane permease, a channel former or a channel blocker, such as, a cecropin-β lytic peptide analog (Jaynes et al. (1993)) which renders transgenic tobacco plants resistant to Pseudomonas solanacearum.

(M) A viral protein or a complex polypeptide derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al. (1990).

(N) An insect-specific antibody or an immunotoxin derived therefrom. Thus, an antibody targeted to a critical metabolic function in the insect gut would inactivate an affected enzyme, killing the insect. For example, Taylor et al. (1994) shows enzymatic inactivation in transgenic tobacco via production of single-chain antibody fragments.

(O) A virus-specific antibody. See, for example, Tavladoraki et al. (1993), which shows that transgenic plants expressing recombinant antibody genes are protected from virus attack.

(P) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al. (1992)). The cloning and characterization of a gene which encodes a bean endopolygalacturonase-inhibiting protein is described by Toubart et al. (1992).

(Q) A developmental-arrestive protein produced in nature by a plant, such as the barley ribosome-inactivating gene, have increased resistance to fungal disease (Longemann et al. (1992)).

2. Genes that Confer Resistance or Tolerance to a Herbicide

(A) A herbicide that inhibits the growing point or meristem, such as an imidazalinone or a sulfonylurea. Exemplary genes in this category code for mutant ALS (Lee et al. (1988)) and AHAS enzyme (Miki et al. (1990)).

(B) Glyphosate (resistance imparted by mutant EPSP synthase and aroA genes) and other phosphono compounds such as glufosinate (PAT and bar genes), and pyridinoxy or phenoxy proprionic acids and cyclohexones (ACCase inhibitor encoding genes). See, for example, U.S. Pat. No. 4,940,835, which discloses the nucleotide sequence of a form of EPSP synthase which can confer glyphosate resistance. A DNA molecule encoding a mutant aroA gene can be obtained under ATCC accession number 39256, and the nucleotide sequence of the mutant gene is disclosed in U.S. Pat. No. 4,769,061. European patent application No. 0 333 033 and U.S. Pat. No. 4,975,374 disclose nucleotide sequences of glutamine synthase genes which confer resistance to herbicides such as L-phosphinothricin. The nucleotide sequence of a phosphinothricin acetyltransferase gene is provided in European application No. 0 242 246. De Greef et al. (1989) describes the production of transgenic plants that express chimeric bar genes coding for phosphinothricin acetyltransferase activity. Exemplary of genes conferring resistance to phenoxy proprionic acids and cyclohexones, such as sethoxydim and haloxyfop, are the Acc1-S1, Acc1-S2 and Acc1-S3 genes described by Marshall et al. (1992).

(C) A herbicide that inhibits photosynthesis, such as a triazine (psbA and GST genes) and a benzonitrile (nitrilase gene). Przibilla et al. (1991) describes the use of plasmids encoding mutant psbA genes to transform Chlamydomonas. Nucleotide sequences for nitrilase genes are disclosed in U.S. Pat. No. 4,810,648, and DNA molecules containing these genes are available under ATCC accession numbers 53435, 67441 and 67442. Cloning and expression of DNA coding for a GST (glutathione S-transferase) is described by Hayes et al. (1992).

3. Genes that Confer Resistance or Tolerance to Environmental Stresses

(A) Cold, freezing or frost. This includes genes that code for proteins that protect from freezing and for enzymes that synthesize cryoprotective solutes. Examples of such genes are Arabidopsis COR15a (Artus et al. (1996)) and spinach CAP160 (Kaye et al. (1998)). Also in this category are regulatory genes that control the activity of other cold tolerance genes (Tomashow and Stockinger (1998)).

(B) Drought or water stress. Kasuga et al. (1999) report how stress inducible expression of DREB1A in trangenic plants increases their tolerance of drought stress. Pilin-Smits et al. (1998) report that expression of baterial genes for synthesis of trehalose produces tolerance of water stress in transgenic tobacco.

(C) Salinity or salt stress. Genes that code for proteins that minimize uptake of sodium in the presence of high salt, or cause the plant to sequester sodium in vacuoles, can enable plants to tolerate higher levels of salt in the soil. The wheat HKT1 potassium transporter, described by Rubio et al. (1999), is an example of the former. Apse et al. (1999) describe how an Arabidopsis Na⁺/H⁺ antiporter can act in the latter manner.

(D) Metals. Protection from the toxic effects of metals such as aluminum and cadmium can be accomplished by transgenic expression of genes that prevent uptake of the metal, or that code for chelating agents that bind the metal ions to prevent them from having a toxic effect. Examples of such genes are Arabidopsis ALR104 and ALR108 (Larsen et al. (1998)) and genes for the enzymes involved in phytochelatin synthesis (Schafer et al. (1998)).

4. Genes that Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al. (1992)).

(B) Decreased phytate content
(1) Introduction of a phytase-encoding gene would enhance breakdown of phytate, adding more free phosphate to the transformed plant, such as the Aspergillus niger phytase gene (Van Hartingsveldt et al. (1993)).
(2) A gene could be introduced that reduces phytate content. In maize, for example, this could be accomplished by cloning and then reintroducing DNA associated with the single allele which is responsible for maize mutants characterized by low levels of phytic acid (Raboy et al. (1990)).

(C) Modified carbohydrate composition effected, for example, by transforming plants with a gene coding for an enzyme that alters the branching pattern of starch. Examples of such enzymes include, Streptococcus mucus fructosyltransferase gene (Shiroza et al. (1988)), Bacillus subtilis levansucrase gene (Steinmetz et al. (1985)), Bacillus licheniformis α-amylase (Pen et al. (1992)), tomato invertase genes (Elliot et al. (1993)), barley amylase gene (Sgaard et al. (1993)), and maize endosperm starch branching enzyme II (Fisher et al. (1993)).

(D) Modified lignin content. The amount or composition of lignin can be altered by increasing or decreasing expression of the biosynthetic enzymes for phenylpropanoid lignin precursors, such as cinnamyl alcohol dehydrogenase (CAD), 4-coumarate:CoA ligase (4CL), and O-methyl transferase (OMT). These and other genes involved in formation of lignin are described in Bloksberg et al. (1998).

5. Selectable Marker Genes:

(A) Numerous selectable marker genes are available for use in plant transformation including, but not limited to, neomycin phosphotransferase II, hygromycin phosphotransferase, EPSP synthase and dihydropteroate synthase. See, Miki et al. (1993).

### EXAMPLES

The present invention is further described by reference to the following Examples, which are offered by way of illustration and are not intended to limit the invention in any manner. Standard techniques well known in the art or the techniques specifically described below were utilized.

### EXAMPLE 1

Generation of Arabidopsis Transgenic Plants by Transformation of Root Explants

A. Agrobacterium Strains and DNA Constructs

Agrobacteria Culture Preparation

All experiments were done with the Agrobacterium tumefaciens strain ABI, which is kanamycin and chloramphenicol resistant. Agrobacteria culture was prepared according to the following steps.
1) A frozen Agrobacterium stock was used to inoculate 10 ml LB liquid medium supplemented with 100 mg/L spectinomycin, 100 mg/L streptomycin, 50 mg/L kanamycin and 25 mg/L chloramphenicol.
2) Cultures were incubated overnight at 28° C. in a shaker at 250 rpm.
3) Cells were collected by centrifugation at 4000×g for 10 min at 4° C.
4) Cells were re-suspended in 10 ml of LB.
5) Steps 3 and 4 were repeated once to remove antibiotics from the medium.
6) Cells were pelleted by centrifugation at 4000×g for 10 min at 4° C.
7) Cells were re-suspended in 30 ml B5 medium (1×B5 salts, 1×B5 vitamins, 3% sucrose, 0.5 g/L MES, pH 5.7). B5 salts and vitamins are described in Gamborg et al. (1968).
   Vector

The binary vector pER16 (FIG. 2) (Zuo et al. (2002a)) was used in root transformation experiments. Because pER16 contains a kanamycin selectable marker for transformed plant cells, after root infection, transgenic plants are selected on SR medium containing kanamycin (1×MS salts, 1% sucrose, 0.5 g/L MES, 1 mg/L 2 isopentyl adenine (2ip), 0.15 mg/L indole acetic acid (IAA), 100 mg/L carbenicillin, 50 mg/L kanamycin, 0.2% Phytagel, pH 5.7).

B. Preparation of Root Explants

Root explants were prepared according to the following steps.
1) Approximately 200 seeds of Arabidopsis thaliana were placed in a 1.5 ml Eppendorf tube.
2) One ml of sterilization solution (50% bleach, 0.001% Triton-100) was added to suspend the seeds.
3) The seed suspension was incubated for 10 minutes, agitating regularly.
4) Seeds were centrifuged for 2 seconds in a table top centrifuge (10,000×g maximum speed).
5) The sterilization solution was aspirated with a sterile pipette.
6) One 1 ml of sterile water was added.
7) Steps 4 to 6 were repeated three times.
8) Seeds were re-suspended in about 0.5 ml sterile agar solution (0.15%) and seeds

germination medium (1×MS salts, 3% sucrose, 8g/l agar, pH5.7). MS salts contain macro-and micronutrients for plant growth and are described in Murashige and Skoog (1962).
9) Seeds were vernalized at 4° C. in the dark for 3 days.
10) The plates were transferred to a tissue culture room at 22° C. with a 16 hr/8 hr light/dark cycle. Seeds germinated in 3-4 days.
11) After two weeks, 15-20 seedlings were transferred to a 250 ml Erlenmeyer flask containing 100 ml B5 medium (1×B5 salts, 1×B5 vitamins, 3% sucrose, 0.5 g/L MES, pH 5.7). The flask opening was covered loosely with two layers of aluminum foil.
12) The flasks were shaken at 125 rpm in a tissue culture room.
13) After 15 days, the seedlings (which should have white colored roots) were transferred into a sterile Petri dish. Roots were separated from aerial parts using a sterile scalpel.
14) The roots were cut into small segments (approximately 1 cm long). Roots should also be scrapped with the scalpel in order to create wounding. This last step increases transformation efficiency.
15) The root segments were placed on four layers of sterile paper towels to drain off excessive medium.
16) Sterile forceps were used to transfer root segments into a 20 mm×100 mm Petri dish containing 40 ml of F1 medium (1×B5 salts, 2% glucose, 0.5 g/L MES, 0.5 mg/L 2,4-D, 0.05 mg/L Kinetin, 0.2% Phytagel, pH 5.7). About 1.5 g -2.0 g of root material can be used per dish. The root explants were spread thoroughly to maximize contact with the medium.
17) The plate was sealed with a gas-permeable tape and incubated under standard culture tissue conditions (22° C., 16 hr/8 hr light/dark cycle) for 2-3 days.
   C. Infection of Root Explants with Agrobacteria

Root explants prepared in (B) were infected with Agrobacteria according to the following steps.
1) Root explants were transferred into a sterile petri dish. A sterile scalpel was used to cut them into approximately 0.5-cm segments.
2) The root explants were mixed with the Agrobacteria solution (A), and the mixture was incubated at room temperature for 5-10 minutes.
3) The explants/Agrobacteria mixture was poured into a sterile basket filter (one layer of cheesecloth). Excessive medium was drained off with several layers of sterile paper towels.
4) Root explants were transferred into a Petri dish containing 40 ml of F2 medium (1×B5 salts, 2% glucose, 0.5 g/L MES, 0.5 mg/L 2,4-D, 0.05 mg/L Kinetin, 0.2% Phytagel, 20 mg/L Acetosyringone, pH 5.7) with a sterile forceps. The root explants can be placed on the F2 medium plate as clumps of 4-10 root segments.
5) The infected root explants were incubated for 2-3 days under standard tissue culture conditions. Upon incubation, explants should be surrounded by a thin layer of Agrobacterium slime but should not be covered by bacteria cells.
6) The infected root explants were transferred and washed in W solution (sterile water, 100 mg/L carbenicillin). Collect the washed root explants by a sterile basket filter.
7) Step 6 was repeated three to four times or until the W solution clarifies. Notes:
3-4 grams of root explants can be infected with 30 ml Agrobacteria solution.

About 1.5-2 grams of root explants can be placed per Petri dish

A wash “basket” filter is a glass tube where an end has been sealed with a mesh or cheesecloth.

D. Regeneration of Transformants and Calculation of Transformation Efficiency

Transformants were regenerated and the regeneration efficiency was calculated according to the following steps.
1) 0.36 g root explants were weighed and then mixed with 10 ml of SR′ medium (1×MS salts, 1% sucrose, 0.5 g/L MES, 1 mg/L 2 ip, 0.15 mg/L IAA, 100 mg/L carbenicillin, 50 mg/L kanamycin, 0.6% lower melting agarose, pH 5.7) which was prewarmed to between 40° C. and 45° C.
2) The mixture was promptly poured into a SR medium plate (1×MS salts, 1% sucrose, 0.5 g/L MES, 1 mg/L 2 ip, 0.15 mg/L IAA, 100 mg/L carbenicillin, 50 mg/L kanamycin, 0.2% Phytagel, pH 5.7) and was well spread by gently swirling and shaking the plate. The plate was sealed with gas-permeable tape and incubated for 2-3 weeks under standard tissue culture conditions (22° C., 16 hr/8 hr light/dark cycle)
3) Shoot regeneration should be apparent after 2-3 weeks. After 40 days (about 6 weeks), shoot number was counted to measure the transformation index (the number of transformed shoots derived from 1 gram of root explants) which is used as a measure of the transformation efficiency.
4) The regenerated shoots were transferred onto a R1 medium (1×MS salts, 1% sucrose, 0.5 g/L MES, 0.15 mg/L IAA, 100 mg/L carbenicillin, 50 mg/L kanamycin, 0.2% Phytagel, pH 5.7) to promote root growth.
5) After 2-3 weeks in culture, roots (see notes) should be sufficiently regenerated. Regenerated plantlets were transferred to soil for T1 seed production. Notes:

Use of 3-5 g explants per 100 ml SR′ medium is recommended.

Approximately 10 ml of root/SR′ mixture should be placed into a SR plate (100 mm×20 mm petri dish containing 50 ml of medium).

Shoots without roots can be transferred into a phytocon containing M medium (1×MS salts, 1×B5 vitamins, 3% sucrose, 100 mg/L carbenicillin, 0.8% Agar, pH 5.7) for adult seedling growth.

### EXAMPLE 2

### Arabidopsis Transformation by Vacuum Infiltration A. Preparation of Plant Material

Plant material was prepared according to the following steps.
1) 10-15 seeds should be used per pot. Seeds were placed in a 15 ml centrifuge tube and re-suspended homogenously with 10 ml of 0.15% agar solution.
2) The tube was left in the dark at 4° C. for 3-4 days (vernalization).
3) Infiltration pots were prepared by putting wet soil in 3.5 in.×3.5 in. square pots covered tightly with a nylon mesh using a rubber band.
4) Using a pipette, the vernalized seeds were distributed evenly on the surface of the pots.
5) The pots were placed in a growth chamber at 22° C. and 8 hr/16 hr light/dark cycle for 4-5 weeks for plant growth. After 4-5 weeks, plants should have healthy-looking rosettes.
6) The pots were transferred to a growth chamber at 22° C. under 16 hr/8 hr light/dark photoperiod for one week.
7) All primary shoots were cut, and the pots were left in the growth chamber under long days for an additional week. This step stimulates lateral shoot formation and therefore the number of flower buds.
   B. Preparation of Agrobacterium Infiltration Solution

An Agrobacterium infiltration solution was prepared according to the following steps.
1) A 250 ml flask containing 20 ml LB medium supplemented with 100 mg/L spectinomycin, 50 mg/L kanamycin and 25 mg/L chloraphenicol was inoculated with Agrobacteria and cultured over night on a shaker (225 rpm) at 28° C.
2) The culture was transferred to a 1000-ml flask containing 250 ml LB medium with 100 mg/L spectinomycin and cultured for 4-6 hours on a shaker (225 rpm) at 28° C.
3) The Agrobacteria were harvested by centrifugation (4000×g for 15 min) and the cell pellet was re-suspended in 300 ml of infiltration solution (5% Sucrose, 10 mM_MgCl₂, 44 nM BAP, 0.005% Silwet L-77). This Agrobacteria infiltration solution was transferred into a 300 ml beaker.
   C. Infiltration Treatment

Plant material was treated for Agrobacterium infiltration according to the following steps.

Pots ready for infiltration (step 7 from A) were inverted into a beaker containing the Agrobacteria infiltration solution (step 4 from B)
2) The vacuum container was closed, and apply vacuum was applied (5-7 mm Hg) for 15 min.
3) After the vacuum treatment, the pots were covered overnight with a saran wrap.
4) The pots were transferred to a growth chamber (22° C., 16 hr/8 hr light/dark cycle) for plant growth and seed production.
5) Seeds were collected when the plants are dry.
   D. Selection of Transformants

Transformants were selected according to the following steps.
1) Seeds (step 5 from C) were incubated in 70% ethanol for 1 minute.
2) The ethanol was removed and sterilization solution (50% bleach, 0.001% Triton-100) was added. The solution was incubated for 10 minutes with constant agitation.
3) The sterilization solution was removed, the seeds were washed three time with sterile water.
4) The seeds were re-suspended in 0.15% sterile agar solution. The seed/agar solution was spread evenly on the surface of growth plates containing selection medium with the appropriate antibiotics for selection (4.3 g/L MS salts, 20 g/L sucrose, 50 mg/L kanamycin, 200 mg/L carbenicillin, 8 g/L agar, pH 5.7).
5) Plates were incubated in a tissue culture room (constant light, 100 μmol/m²/sec)
6) After 10 days, transformants (transformed seedlings are green whereas untransformed ones are white) were selected and transferred to soil for seed production.

### EXAMPLE 3

### Bioassays of Shoot Regeneration frequency from Arabidopsis Root Explants

Shoots were regenerated from Arabidopsis root explants according to the following steps.
1) Steps1-17 of B in Example 1 for the preparation of root explants were followed.
2) After 4-5 days culture in F1 medium, the root explants were transferred in a sterile petri dish and were cut into 0.5 cm segment by using a sterile scalpel.
3) 0.2 gram of root explants was mixed with 10 ml SI′ medium (1×MS salts, 1% sucrose, 0.5 g/L MES, 1 mg/L 2 ip, 0.15 mg/L IAA, 0.6% lower melting agarose, pH 5.7) pre-warmed to between 40° C. and 45° C.
4) The mixture was promptly poured into a SI medium plate (1×MS salts, 1% sucrose, 0.5 g/L MES, 1 mg/L 2 ip, 0.15 mg/L IAA, 0.2% Phytagel, pH 5.7) and spread well by gently swirling and shaking the plate. The plate was sealed with gas-permeable tape and incubated in a tissue culture room.
5) After 3-4 weeks culture, the shoot number per plate was counted and the shoot regeneration index (shoots number per 1 gram of roots explants) was calculated, which is used to express shoots regeneration efficiency.

### EXAMPLE 4

### Arabidopsis thaliana Transformation Efficiency is Dependent on ABI5 Protein Levels

A. Transformation by Vacuum Infiltration

Agrobacterium tumefaciens (abi strain) carrying the pER16 vector was used to transform Arabidopsis using the vacuum infiltration approach (as described in Example 2). This vector confers kanamycin resistance to transgenic seedlings which can be easily distinguished from untransformed seedlings after 10 days of culture on the selection medium.

Plants of WT (Ws ecotype), abi5-4 (Ws ecotype) and WT carrying a 35S-ABI5 transgene (Ws ecotype) were infilltrated. In each case, one thousand seedlings were assessed to measure transformation efficiency. In the case of abi5-4 mutants, 8 independent experiments were performed. Table 1 shows the percentage of transformation in each genetic background.

TABLE 1
Transformation Efficiency¹

| Wild type | abi5-4 | 35S-ABI5 |
|---|---|---|
| 0.7% | 5.9% | 0.2% |
| 1% | 47.6% | 0.4% |
| 1.5% | 5.8% | 0.2% |
| 0.5% | 8.8% | |
| | 5.0% | |
| | 2.3% | |
| | 2% | |
| | 6.5% | |
| Average: 0.93% | Average: 10.5% | Average: 0.27% |

¹values for each independent experiment and average

These results demonstrate that there is a direct correlation between ABI5 protein levels and efficiency of transformation.

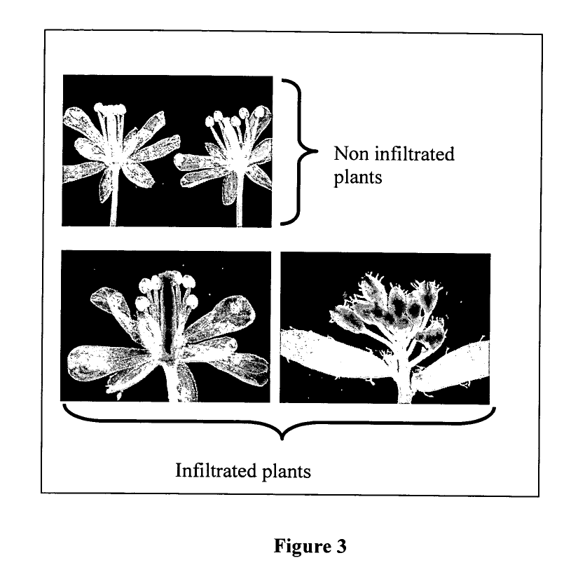

Because ovules have been reported to be the target tissue for Agrobacterium transformation (Ye et al., 1999), ABI5 expression in floral tissue was examined during and after vacuum infiltration with Agrobacterium. For this purpose, Arabidopsis transgenic plants carrying ABI5 promoter sequences driving the expression of the ABI5 open reading frame fused to the marker gene GUS was used. Before vacuum infiltration, ABI5 express in ovules tissue. In FIG. 3, there are two flowers before vacuum infiltration. The left one is the flower from transgenic plants carrying ABI5 promoter driving GUS gene and shows the GUS signal in ovules tissue. The right one is wild type control. The GUS signal of flowers after vacuum infiltration in FIG. 3 clearly shows that ABI5::GUS expression was induced by the vacuum infiltration treatment. This induction was dependent on the presence of Agrobacterium cells in the infiltration medium. These results are consistent with the notion that ABI5 levels in Agrobacterium target tissues inhibit transformation efficiency.

B. Transformation of Root Explants

Root explants transformation in Arabidopsis is widely used in plant molecular biology research because this method is robust and produces high transformation efficiency. Transformation efficiency can be measured using the shoot regeneration index, which is defined as the number of transformed shoots per one gram of roots explants.

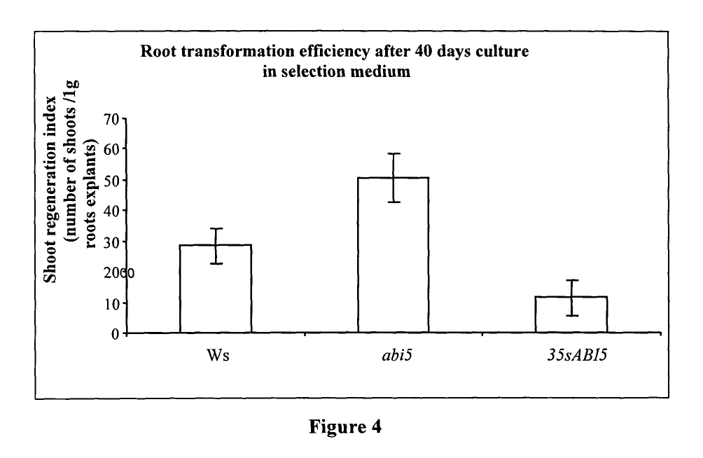

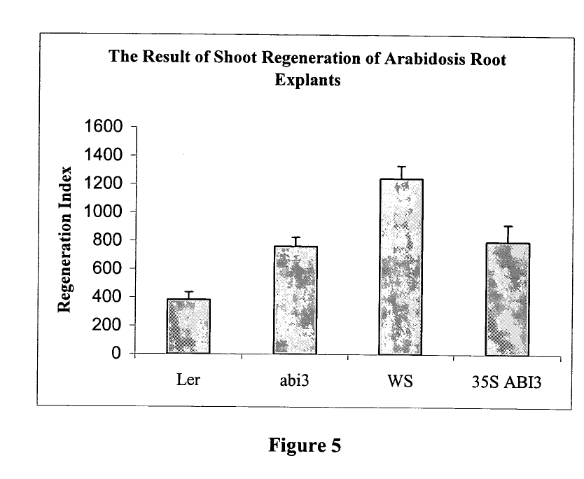

Wild type, abi5-4 and 35S-ABI5 plant materials were used as sources of root explants for transformation (as described in Example 1). pER16 was used as a vector for root transformation. After culture in the selection medium for 40 days, the transformation efficiency of abi5-4 was measured to be 50.7, whereas the wild type one was found to be 28.31 (FIG. 4). By contrast, 35S-ABI5 root explains an gave an index of 11.53 (FIG. 4). These results were obtained from four independents experiments. In conclusion, the data demonstrate that absence of ABI5 increases stable transformation in root explants whereas ABI5 over expression inhibits it.

Taken together, these results indicate that down regulating ABI5 levels or its activity facilitates the frequency of stable transformation mediated by Agrobacterium tumefaciens in Arabidopsis.

### EXAMPLE 5

### Shoot Regeneration from Root Explants is Inversely Correlated with ABI5 Protein Levels

A prerequisite for high transformation efficiency from root explants is to have high shoot regeneration. One possible explanation for the higher transformation efficiency in abi5 mutants may be related to shoot regeneration efficiency. To examine shoot regeneration efficiency as a function of ABI5 protein levels, shoot regeneration in wild type (WT) (Ws ecotype) abi5-4 (Ws ecotype) and transgenic WT (Ws ecotype) carrying a 35S-ABI5 was measured. As shown, in Table 2, it was found that, compared to WT plants, shoot regeneration efficiency was markedly reduced in 35S-ABI5.

TABLE 2
Shoot Regeneration Efficiencies¹

| | WT (WS ecotype) | abi5-4 | abi5-1 | 35S-ABI5 |
|---|---|---|---|---|
| Experiment 1 | 1175 | 2600 | 2380 | 675 |
| | 1522 | 2515 | 2250 | 600 |
| | 1150 | 2650 | 2090 | 670 |
| | 1170 | 2800 | 2050 | 680 |
| | 1040 | 2540 | 2040 | 675 |
| | 1220 | 2650 | 2000 | 680 |
| | 1180 | 2600 | 2030 | 675 |
| | 1250 | 2510 | 1990 | 515 |
| | 1100 | 2570 | 2005 | 550 |
| Averages | 1201 | 2604 | 2092 | 636 |
| Experiment 2 | 1266 | 2340 | 1933 | 517 |
| | 1426 | 2667 | 2253 | 693 |
| | 1040 | 2840 | 1947 | 500 |
| | 1453 | 2533 | 2267 | 547 |
| | 1306 | 2343 | 2213 | 633 |
| | 1227 | 2693 | 2160 | 673 |
| | 1347 | 2667 | 2133 | 707 |
| | 1160 | 2933 | 2263 | 714 |
| | 1333 | 2706 | 2233 | 540 |
| Averages | 1284 | 2635 | 2155 | 614 |

¹shoot number per gram of root explant material

### EXAMPLE 6

### Shoot Regeneration from Explants is Inversely Correlated with AB13 Levels

The model plant Arabidopsis thaliana has several ecotypes. In this experiment, two different ecotypes, Lansberg erecta (Ler) and Wassilewskaja (WS) were used. The abi3 mutant is a null mutant in the ABI3 gene in the Ler background, whereas the 35S-ABI3 over-expressor is in the WS background. Therefore, appropriate wild type (WT) controls were used.

Root explains of Arabidopsis WT Ler, abi3 (in Ler background), WT/WS and 35S ABI3/WS tranagenic line were pre-cultured in F1 medium for 4 days and then transferred to shoot regenerate shoots (SI) medium to regenerate shoots. 0.2 g of root explants were cultured on each plate containing 50 ml SI medium. A total of 9 plates were used for each genotype. After 20 days of culture, the number of shoots regenerated per gram of fresh root explants (Shoot Regeneration Index) was determined. This number used to indicate the ability of root segments to regenerate shoots. This results are shown in FIG. 5.

Shoot regenerate efficiency of abi3 is about 2 times higher than that of WT Ler control. In the same generic backgound of Arabidopsis thaliana (WS ecotype), the shoot regenerate efficiency of 35S-ABI3 is lower than that of WS.

These results show that the shoot regeneration efficiency from Arabidopsis root explants is inversely related to ABI3 expression levels. Low ABI3 expression levels increase regeneration frequency whereas high ABI3 expression levels reduce regeneration frequency. Since ABI3 controls the expression of ABI5 (Lopez-Molina et al., 2002b), the changes in regenerated frequency are likely related to the effect of ABI3 on ABI5 levels. This example demonstrates that methods which modulate the expression of ABI5 also enhance regeneration efficiency.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### Bibliography

Abe et al. 1987. J. Biol. Chem. 262:16793.
Apse, M. P. et al. 1999. Science 285:1256-1258.
Artus, N. N. et al. 1996. Proc. Natl. Acad. Sci USA 93:13404-13409.
Beachy, et al. 1990. Ann. Rev. Phytopathol. 28:451.
Bensmihen, S., S. Rippa, G. Lambert, D. Jublot, V. Pautot, F. Granier, J. Giraudat, and F. Parcy. 2002. The homologous ABI5 and EEL transcription factors function antagonistically to fine-tune gene expression during late embryogenesis. Plant Cell 14: 1391-403.
Bloksberg, L. N. et al. 1998. U.S. Pat. No. 5,850,020.
Bobb, A. J., H. G. Eiben, and M. M. Bustos. 1995. PvAlf, an embryo-specific acidic transcriptional activator enhances gene expression from phaseolin and phytohemagglutinin promoters. Plant J 8:331-43.
Botella, et al. 1994. Plant Molec. Biol. 24:757.
Cahoon, R. E., G. Lu, and M. E. Williams. 2003. Plant Transcription Factors, U.S. Published Patent Application No. 2003/0119165 A1.
Choi, H., J. Hong, J. Ha, J. Kang, and S. Y. Kim. 2000. ABFs, a family of ABA-responsive element binding factors. J Biol Chem 275:1723-30.
Chua, N. H. and T. Aoyama. 2000. Chemical inducible promoters used to obtain transgenic plants with silent marker. U.S. Pat. No. 6,063,985.
Chuang, C. F. and Meyerowitz, E. M. 2000. Specific and heritable genetic interference by double-stranded RNA in Arabidopsis thaliana. Proc. Natl. Acad. Sci. USA 97:4985-4990
Davies, C. and S. P. Robinson. 2000. Differential screening indicates a dramatic change in mRNA profiles during grape berry ripening. Cloning and characterization of cDNAs encoding putative cell wall and stress response proteins. Plant Physiol 122:803-12.
Elliot, et al. 1993. Plant Molec. Biol. 21:515.
Finkelstein, R. R., S. S. Gampala, and C. D. Rock. 2002. Abscisic acid signaling in seeds and seedlings. Plant Cell 14 Suppl:S15-45.
Finkelstein, R. R. and T. J. Lynch. 2000. The Arabidopsis abscisic acid response gene ABI5 encodes a basic leucine zipper transcription factor. Plant Cell 12:599-609.
Finkelstein R, R., M. L. Wang, T. J. Lynch, S. Rao, and H. M.Goodman. 1998. The Arabidopsis abscisic acid response locus ABI4 encodes an APETALA 2 domain protein. Plant Cell 10:1043-1054.
Finkelstein, R. R. 1994. Mutations at two new Arabidopsis ABA response loci are similar to the abi3 mutations. Plant J 5:765-771.
Fisher, et al. 1993. Plant Physiol. 102:1045.
Gamborg, O. L. et al. 1968. Exp. Cell Res. 50:151.
Geiser, et al. 1986. Gene 48:109.
Giraudat, J., B. M. Hauge, C. Valon, J. Smalle, F. Parcy, and H. M. Goodman. (1992). Isolation of the Arabidopsis AB13 gene by positional cloning. Plant Cell 4:1251-1261.
Gosti, F., N. Beaudoin, C. Serizet, A. A. Webb, N. Vartanian, and J. Giraudat. 1999. ABI1 protein phosphatase 2C is a negative regulator of abscisic acid signaling. Plant Cell 11:1897-1910.
Griess, et al. 1994. Plant Physiol. 104:1467.
Guo, H.-S., Fei, J.-F., Xie, Q., Chua, N.-H. 2003 A chemical-regulated inducible RNAi system in plants. Plant J 34:383-392
Hayes, et al. 1992. Biochem. J. 285:173.
Himmelbach, A., M. Iten and E. Grill. 1998. Signalling of abscisic acid to regulate plant growth. Philos. Trans. R. Soc. Lond. B Biol. Sci. 353:1439-1444.
Hobo, T., Y. Kowyama, and T. Hattori. 1999. A bZIP factor, TRAB1, interacts with VP1 and mediates abscisic acid-induced transcription. Proc Natl Acad Sci USA 96:15348-53. Horvath, D. M., N. H. Chua, M. H. Stuiver and I. Jepson. 2002. Salicyclic acid inducible genes and promoters. U.S. Published Patent Application No. 2002/0039780 A1.
Huub, et al. 1993. Plant Molec. Biol 21:985.
Jakoby, M., B. Weisshaar, W. Droge-Laser, J. Vicente-Carbajosa, J. Tiedemann, T. Kroj, and F. Parcy. 2002. bZIP transcription factors in Arabidopsis. Trends Plant Sci 7:106-11.
Jaynes, et al. 1993. Plant Sci. 89:43.
Johnson, R. R., R. L. Wagner, S. D. Verhey, and M. K. Walker-Simmons. 2002. The abscisic acid-responsive kinase PKABA1 interacts with a seed-specific abscisic acid response element-binding factor, TaABF, and phosphorylates TaABF peptide sequences. Plant Physiol 130:837-46.
Jones, et al. 1994. Science 266:789.
Kasuga, M. et al. 1999. Nature Biotech. 17:287-291.
Kawalleck, et al. 1993. Plant Molec. Biol. 21:673.
Kaye, C. et al. 1998. Plant Physiol. 116:1367-1377.
Kim, S. Y., H. J. Chung, and T. L. Thomas. 1997. Isolation of a novel class of bZIP transcription factors that interact with ABA-responsive and embryo-specification elements in the Dc3 promoter using a modified yeast one-hybrid system. Plant J 11: 1237-51.
Kim, S. Y., J. Ma, P. Perret, Z. Li, and T .L. Thomas. 2002. Arabidopsis ABI5 subfamily members have distinct DNA-binding and transcriptional activities. Plant Physiol 130:688-97.
Knultzon, et al. 1992. Proc. Nat. Acad. Sci. USA 89:2624.
Kornneef, M., G. Reuing and C. M. Karssen. 1984. The isolation and characterization of abscisic acid-insensitive mutants of Arabidopsis thaliana. Physiol Plant 61:377-383.
Kramer, et al. 1993. Insect Molec. Biol. 23:691.
Lamb, et al. 1992. Bio/Technology 10:1436.
Larsen, P. B. et al. 1998. Plant Physiol. 117:9-18.
Lazarova, G., Y. Zeng, and A. R. Kermode. 2002. Cloning and expression of an ABSCISIC ACID-INSENSITIVE 3 (ABI3) gene homologue of yellow-cedar (Chamaecyparis nootkatensis). J Exp Bot 53:1219-21.
Lee, et al. 1988. EMBO J. 7:1241.
Leung, J., M. Bouvier-Durand, P. C. Morris, D. Guerrier, F. Chefdor, and J. Giraudat. 1994. Arabidopsis ABA response gene ABI1: features of a calcium-modulated protein phosphatase. Science 264:1448-1452.
Longemann, et al. 1992. Bio/Technology 10:3305.
Lopez-Molina, L. and N. H. Chua. 2000. A null mutation in a bZIP factor confers ABA-insensitivity in Arabidopsis thaliana. Plant Cell Physiol 41: 541-7.
Lopez-Molina, L., S. Mongrand, and N. H. Chua. 2001. A postgermination developmental arrest checkpoint is mediated by abscisic acid and requires the ABI5 transcription factor in Arabidopsis. Proc Natl Acad Sci USA 98:4782-7.
Lopez-Molina, L., S. Mongrand, and N. H. Chua. 2002a. Overexpression of ABI5 in plants to prevent precocious seed germination and to confer resistance to drought and high salt. U.S. Published Patent Application 2002/0174454 A1.
Lopez-Molina, L., S. Mongrand, D. T. McLachlin, B. T. Chait and N. H. Chua. 2002b. ABI5 acts downstream of ABI3 to execute an ABA-dependent growth arrest during germination. Plant J. 32:317-328.
Lopez-Molina, L., S. Mongrand, N. Kinoshita and N. H. Chua. 2003. AFP is a novel negative regulator of ABA signaling that promotes ABI5 protein degradation. Genes Develop 17:410-418.
Marshall, et al. 1992. Theor. Appl. Genet. 83:435.
Martin, et al. 1993. Science 262:1432.
Meyer, K., M. P. Leube, and E. Grill. 1994. A protein phosphatase 2C involved in ABA signal transduction in Arabidopsis thaliana. Science 264:1452-1455.
Miki, et al. 1990. Theor. Appl. Genet. 80:449.
Miki, et al. 1993. “Procedures for Introducing Foreign DNA into Plants,” in Methods in Plant Molecular Biology and Biotechnology, Glick et al. (eds.), CRC Press, pp. 67-88.
Mindrinos, et al. 1994. Cell 78:1089.
Moller, S., J. Zuo and N. H. Chua. 2001. Inducible site-specific recombination for the activatoin and removal of transgenes in transgenic plants. International Published Patent Application No. WO 01/40492.
Murashige, T. and Skoog, F. 1962. Physiol. Plant. 15:473.
Needleman, S. B., and C. D. Wunsch. 1970. A general method applicable to the search for similarities in the amino acid sequence of two proteins. J. Mol. Biol. 48:443-453.
Noel, F., Kissen, R. and Du Jardin, P. 2003. Molecular cloning and characterization of genes homologous to the Vpl-Abi3 genes family. Genbank Accession Nos. AJ309217 and AJ309218.
Pang, et al. 1992. Gene 116:165.
Parcy, F. and J. Giraudat. 1997. Interactions between the ABI1 and the ectopically expressed ABI3 genes in controlling abscisic acid responses in Arabidopsis vegetative tissues. Plant J11:693-702.
Pen, et al. 1992. Bio/Technology 10:292.
Pilin-Smits, E. A. H. et al. 1998. J Plant Physiol. 152:525-532.
Pratt, G. E. et al. 1989. Biochem Biophys Res Commun. 163:1243-7.
Przibilla, et al. 1991. Plant Cell 3:169.
Raboy, et al. 1990. Maydica 35:383.
Regan, 1994. J. Biol. Chem. 269:9.
Rohde, A., E. Prinsen, R. De Rycke, G. Engler, M. Van Montagu, and W. Boedjan. 2002. PtABI3 impinges on the growth and differentiation of embryonic leaves during bud set in poplar. Plant Cell 14:1885-901. Erratum in: 2002. Plant Cell 14:2975.
Rubio, F. et al. 1999. J. Biol Chem. 274:6839-47.
Sano, T. and T. Nagata. 2002. The possible involvement of a phosphate-induced transcription factor encoded by phi-2 gene from tobacco in ABA-signaling pathways. Plant Cell Physiol 43:12-20.
Schafer, H. J. et al. 1998. Plant Mol. Biol. 37:87-97.
Shiota, H., R. Satoh, K. Watabe, H. Harada, and H. Kamada. 1998. C-ABI3, the carrot homologue of the Arabidopsis ABI3, is expressed during both zygotic and somatic embryogenesis and functions in the regulation of embryo-specific ABA-inducible genes. Plant Cell Physiol 39:1184-93.
Shiroza, et al. 1988. J Bacteriol. 170:810.
Shuman, J. D., C. R. Vinson, and S. L. McKnight. 1990. Evidence of changes in protease sensitivity and subunit exchange rate on DNA binding by C/EBP. Science 249: 771-4.
Smith and Waterman. 1981. Advances in Applied Mathematics 2:482-489.
Sogaard, et al. 1993. J. Biol. Chem. 268:22480.
Steinmetz, et al. 1985. Mol. Gen. Genet. 200:220.
Sumitani, et al. 1993. Biosci. Biotech. Biochem. 57:1243.
Tavladoraki, et al. 1993. Nature 266:469.
Taylor, et al. 1994. Abstract #497, Seventh Int'l. Symposium on Molecular Plant-Microbe Interactions.
Tavladoraki, et al. 1993. Nature 266:469.
Tomashow, M. F. and E. J. Stockinger. 1998. PCT International Publication Number WO 98/09521.
Toubart, et al. 1992. Plant J 2:367.
Uno, Y., T. Furihata, H. Abe, R. Yoshida, K. Shinozaki, and K. Yamaguchi-Shinozaki. 2000. Arabidopsis basic leucine zipper transcription factors involved in an abscisic acid-dependent signal transduction pathway under drought and high-salinity conditions. Proc Natl Acad Sci USA 97:11632-7.
Van Damme, et al. 1994. Plant Molec. Biol. 24:825.
Van Hartingsveldt, et al. 1993. Gene 127:87. Vinson, C. R., P. B. Sigler, and S. L. McKnight. 1989. Scissors-grip model for DNA recognition by a family of leucine zipper proteins. Science 246:911-6.
Waterhouse, P. M., Graham, M. W. and Wang, M. B. 1998 Virus resistance and gene silencing in plants can be induced by simultaneous expression of sense and antisense RNA. Proc. Natl. Acad. Sci. USA 95:13959-13964
Ye G. N., D. Stone, S. Z. Pang. W. Creely, K. Gonzalez and M. Hinchee 1999. Arabidopsis ovule is the target for Abrobacterium in planta vauum infiltration transformation. Plant J. 19:249-57.
Zuo, J. Q. W. Niu, S. G. Mller and N. H. Chua. 2001. Chemical-regulated, site-specific DNA excision in transgenic plants. Nature Biotechnology 19:157-161.
Zuo, J., Q. W. Niu, G. Frugis and N. H. Chua. 2002a. The WUSCHEL gene promotes vegetative-to-embryonic transition in Arabidopsis. Plant J 30:349-59.
Zuo, J., Q. W. Niu and N. H. Chua. 2002b. Chemical inducible promoters used to obtain transgenic plants with silent marker. U.S. Pat. No. 6,452,068 B 1.
Zuo, J., Q. W. Niu and N. H. Chua. 2003. Chemical inducible promoters used to obtain transgenic plants with silent marker. U.S. Published Patent Application No. 2003/0150013 A1.

1. A method for enhancing the frequency for producing transgenic plants by Agrobacterium-mediated transformation which comprises incubating plant cells with Agrobacterium under conditions in which the protein level or activity of ABI5, ABI5 homolog or ABI ortholog in the plant cells is downregulated to produce Agrobacterium-mediated transformed plant cells and regenerating transgenic plants from the transformed plant cells, wherein the downregulation of the protein level or activity of ABI5, ABI5 homolog or ABI5 ortholog results in an enhanced frequency of transgenic plant production.

2. The method of claim 1, wherein the plant cells are obtained from a transgenic plant having a downregulated protein level or activity of ABI5, ABI5 homolog or ABI5 ortholog to provide the downregulation condition.

3. The method of claim 2, wherein the downregulation is under control of a gene cassette for the activation and removal of a nucleic acid sequence in a transgenic plant, wherein said nucleic acid sequence downregulates said protein level or activity.

4. The method of claim 2, wherein the transgenic plant comprises a T-DNA insertion in an ABI5, ABI5 homolog or ABI5 ortholog gene to downregulate the ABI5, ABI5 homolog or ABI5 ortholog.

5. The method of claim 2, wherein the transgenic plant comprises a transposon insertion in an ABI5, ABI5 homolog or ABI5 ortholog gene to downregulate the ABI5, ABI5 homolog or ABI5 ortholog.

6. The method of claim 2, wherein the transgenic plant comprises an interfering RNA to downregulate the ABI5, ABI5 homolog or ABI5 ortholog.

7. The method of claim 2, wherein the transgenic plant comprises an overexpressed negative regulator of ABI5, ABI5 homolog or ABI ortholog to downregulate the ABI5, ABI5 homolog or ABI5 ortholog.

8. The method of claim 2, wherein the transgenic plant comprises a downregulated protein level or activity of a protein that modulates expression of ABI5, ABI5 homolog or ABI5 ortholog to downregulate the ABI5, ABI5 homolog or ABI5 ortholog.

9. The method of claim 8, wherein the transgenic plant comprises a T-DNA insertion in the modulating protein to downregulate the modulating protein.

10. The method of claim 8, wherein the transgenic plant comprises a transposon insertion in the modulating protein to downregulate the modulating protein.

11. The method of claim 8, wherein the transgenic plant comprises an interfering RNA to downregulate the modulating protein.

12. The method of claim 8, wherein the transgenic plant comprises an overexpressed negative regulator of the modulating protein to downregulate the modulating protein.

13. The method of claim 8, wherein the modulating protein is ABI3, ABI3 homolog or ABI3 ortholog.

14. The method of claim 1, wherein the plant cells are incubated with a plant vector which comprises a DNA sequence which downregulates ABI5, ABI5 homolog or ABI5 ortholog to produce the downregulated condition.

15. The method of claim 14, wherein the DNA sequence is located in a gene cassette for the activation and removal of the DNA sequence in a transgenic plant.

16. The method of claim 14, wherein the DNA sequence comprises ABI5, ABI5 homolog or ABI5 ortholog lacking an activation domain such that inactive heterodimers are formed between the DNA sequence and the endogenous wild type ABI5, ABI5 homolog or ABI5 ortholog to downregulate ABI5, ABI5 homolog or ABI5 ortholog.

17. The method of claim 14, wherein the DNA sequence produces a hairpin RNA sequence homologous to ABI5, ABI5 homolog or ABI5 ortholog to downregulate ABI5, ABI5 homolog or ABI5 ortholog.

18. The method of claim 14, wherein the DNA sequence downregulates a protein level or activity of a protein that modulates expression of ABI5, ABI5 homolog or ABI5 ortholog to downregulate the ABI5, ABI5 homolog or ABI5 ortholog.

19. The method of claim 18, wherein the DNA sequence comprises the modulating protein lacking an activation domain such that inactive heterodimers are formed between the DNA sequence and the protein modulator to downregulate ABI5, ABI5 homolog or ABI5 ortholog.

20. The method of claim 18, wherein the DNA sequence produces a hairpin RNA sequence homologous to the modulating protein to downregulate ABI5, ABI5 homolog or ABI5 ortholog.

21. The method of claim 18, wherein the modulating protein is ABI3, ABI3 homolog or ABI3 ortholog.

22. A method for enhancing the frequency for regenerating transgenic plants which comprises transforming plant cells to produce transformed plant cells and regenerating transformed plant cells, wherein the protein level or activity of ABI5, ABI5 homolog or ABI ortholog in the plant cells is downregulated to wherein the downregulation of the protein level or activity of ABI5, ABI5 homolog or ABI5 ortholog results in an enhanced frequency of regenerated transgenic plants.

23. The method of claim 22, wherein the plant cells are obtained from a transgenic plant having a downregulated protein level or activity of ABI5, ABI5 homolog or ABI5 ortholog to provide the downregulation condition.

24. The method of claim 22, wherein the downregulation is under control of a gene cassette for the activation and removal of a nucleic acid sequence in transgenic plant, wherein said nucleic acid sequence downregulates said protein level or activity.

25. The method of claim 23, wherein the transgenic plant comprises a T-DNA insertion in an ABI5, ABI5 homolog or ABI5 ortholog gene to downregulate the ABI5, ABI5 homolog or ABI5 ortholog.

26. The method of claim 23, wherein the transgenic plant comprises a transposon insertion in an ABI5, ABI5 homolog or ABI5 ortholog gene to downregulate the ABI5, ABI5 homolog or ABI5 ortholog.

27. The method of claim 23, wherein the transgenic plant comprises an interfering RNA to downregulate the ABI5, ABI5 homolog or ABI5 ortholog.

28. The method of claim 23, wherein the transgenic plant comprises an overexpressed negative regulator of ABI5, ABI5 homolog or ABI ortholog to downregulate the ABI5, ABI5 homolog or ABI5 ortholog.

29. The method of claim 23, wherein the transgenic plant comprises a downregulated protein level or activity of a protein that modulates expression of ABI5, ABI5 homolog or ABI5 ortholog to downregulate the ABI5, ABI5 homolog or ABI5 ortholog.

30. The method of claim 29, wherein the transgenic plant comprises a T-DNA insertion in the modulating protein to downregulate the modulating protein.

31. The method of claim 29, wherein the transgenic plant comprises a transposon insertion in the modulating protein to downregulate the modulating protein.

32. The method of claim 29, wherein the transgenic plant comprises an interfering RNA to downregulate the modulating protein.

33. The method of claim 29, wherein the transgenic plant comprises an overexpressed negative regulator of the modulating protein to downregulate the modulating protein.

34. The method of claim 29, wherein the modulating protein is ABI3, ABI3 homolog or ABI3 ortholog.

35. The method of claim 22, wherein the plant cells are incubated with a plant vector which comprises a DNA sequence which downregulates ABI5, ABI5 homolog or ABI5 ortholog to produce the downregulated condition.

36. The method of claim 35, wherein the DNA sequence is located in a gene cassette for the activation and removal of the DNA sequence in a transgenic plant.

37. The method of claim 35, wherein the DNA sequence comprises ABI5, ABI5 homolog or ABI5 ortholog lacking an activation domain such that inactive heterodimers are formed between the DNA sequence and the endogenous wild type ABI5, ABI5 homolog or ABI5 ortholog to downregulate ABI5, ABI5 homolog or ABI5 ortholog.

38. The method of claim 35, wherein the DNA sequence produces a hairpin RNA sequence homologous to ABI5, ABI5 homolog or ABI5 ortholog to downregulate ABI5, ABI5 homolog or ABI5 ortholog.

39. The method of claim 35, wherein the DNA sequence downregulates a protein level or activity of a protein that modulates expression of ABI5, ABI5 homolog or ABI5 ortholog to downregulate the ABI5, ABI5 homolog or ABI5 ortholog.

40. The method of claim 39, wherein the DNA sequence comprises the modulating protein lacking an activation domain such that inactive heterodimers are formed between the DNA sequence and the protein modulator to downregulate ABI5, ABI5 homolog or ABI5 ortholog.

41. The method of claim 39, wherein the DNA sequence produces a hairpin RNA sequence homologous to the modulating protein to downregulate ABI5, ABI5 homolog or ABI5 ortholog.

42. The method of claim 39, wherein the modulating protein is ABI3, ABI3 homolog or ABI3 ortholog.

43. A transgenic plant comprising a downregulated protein level or activity of ABI5, ABI5 homolog or ABI5 ortholog

44. The transgenic plant of claim 43, wherein the downregulation is under control of a gene cassette for the activation and removal of a nucleic acid sequence in a transgenic plant, wherein said nucleic acid sequence downregulates said protein level or activity.

45. The transgenic plant of claim 43 which comprises a T-DNA insertion in an ABI5, ABI5 homolog or ABI5 ortholog gene to downregulate the ABI5, ABI5 homolog or ABI5 ortholog.

46. The transgenic plant of claim 43 which comprises a transposon insertion in an ABI5, ABI5 homolog or ABI5 ortholog gene to downregulate the ABI5, ABI5 homolog or ABI5 ortholog.

47. The transgenic plant of claim 43 which comprises an interfering RNA to downregulate the ABI5, ABI5 homolog or ABI5 ortholog.

48. The transgenic plant of claim 43 which comprises an overexpressed negative regulator of ABI5, ABI5 homolog or ABI ortholog to downregulate the ABI5, ABI5 homolog or ABI5 ortholog.

49. The transgenic plant of claim 43 which comprises a downregulated protein level or activity of a protein that modulates expression of ABI5, ABI5 homolog or ABI5 ortholog to downregulate the ABI5, ABI5 homolog or ABI5 ortholog.

50. The transgenic plant of claim 49 which comprises a T-DNA insertion in the modulating protein to downregulate the modulating protein.

51. The transgenic plant of claim 49 which comprises a transposon insertion in the modulating protein to downregulate the modulating protein.

52. The transgenic plant of claim 49 which comprises an interfering RNA to downregulate the modulating protein.

53. The transgenic plant of claim 49 which comprises an overexpressed negative regulator of the modulating protein to downregulate the modulating protein.

54. The transgenic plant of claim 49, wherein the modulating protein is ABI3, ABI3 homolog or ABI3 ortholog.

55. A plant vector which comprises a DNA sequence which downregulates ABI5, ABI5 homolog or ABI5 ortholog in a host target plant cell.

56. The plant vector of claim 55, wherein the DNA sequence is located in a gene cassette for the activation and removal of the DNA sequence in a transgenic plant.

57. The plant vector of claim 55, wherein the DNA sequence comprises ABI5, ABI5 homolog or ABI5 ortholog lacking an activation domain such that inactive heterodimers are formed between the DNA sequence and the endogenous wild type ABI5, ABI5 homolog or ABI5 ortholog to downregulate ABI5, ABI5 homolog or ABI5 ortholog.

58. The plant vector of claim 55, wherein the DNA sequence produces a hairpin RNA sequence homologous to ABI5, ABI5 homolog or ABI5 ortholog to downregulate ABI5, ABI5 homolog or ABI5 ortholog.

59. The plant vector of claim 55, wherein the DNA sequence downregulates a protein level or activity of a protein that modulates expression of ABI5, ABI5 homolog or ABI5 ortholog to downregulate the ABI5, ABI5 homolog or ABI5 ortholog.

60. The plant vector of claim 59, wherein the DNA sequence comprises the modulating protein lacking an activation domain such that inactive heterodimers are formed between the DNA sequence and the protein modulator to downregulate ABI5, ABI5 homolog or ABI5 ortholog.

61. The plant vector of claim 59, wherein the DNA sequence produces a hairpin RNA sequence homologous to the modulating protein to downregulate ABI5, ABI5 homolog or ABI5 ortholog.

62. The plant vector of claim 59, wherein the modulating protein is ABI3, ABI3 homolog or ABI3 ortholog.
